(19) United States
(12) Patent Application Publication
(10) Pub. No.: US 2026/0297513 A1
(43) Pub. Date: Oct. 1, 2026

(54) METHOD AND SYSTEMS FOR INNERVATING BIOFABRICATED TISSUES

(71) Applicant: UNIVERSITY OF ROCHESTER, Rochester, NY (US)

(72) Inventors: Hossein ABOLHASSANI, Rochester, NY (US); Lisa DELOUISE, Rochester, NY (US)

(21) Appl. No.: 19/554,091

(22) Filed: Mar. 2, 2026

Related U.S. Application Data
(60) Provisional application No. 63/778,553, filed on Mar. 27, 2025.

(51) Int. Cl. C12N 5/0793; C12N 5/071

(52) U.S. Cl. CPC C12N 5/0619; C12N 5/0633; C12N 5/0688; C12N 2501/13; C12N 2513/00; C12N 2533/52; C12N 2533/54

(57) ABSTRACT

A method for three-dimensional innervation of biofabricated tissue is disclosed. The method comprises the steps of: culturing neurons or neuron progenitor cells on a surface, wherein the surface is treated with an extracellular matrix (ECM) protein and/or oxygen plasma; casting a hydrogel layer on top of the cultured neurons with or without a membrane, wherein the hydrogel layer has a thickness equal to or less than the maximum ability of neurons to vertically extend their neurites in a specific culture period; and culturing a target tissue on top of the hydrogel layer to allow neurites from the differentiated neurons to extend through the hydrogel and innervate the target tissue.

This application claims priority to U.S. Provisional application 63/778,553 filed on Mar. 27, 2025, the contents of which are incorporated herein by reference in their entirety and for all purposes.

This invention was made with government support under DE027695 and DE034549, awarded by the National Institutes of Health. The government has certain rights in the invention.

### FIELD

This application relates to the field of biomedical and tissue engineering and, in particular, to biofabricated innervated tissues.

### BACKGROUND

More than 90% of drug candidates fail in clinical trial stages and cannot gain Food and Drug Administration (FDA) approval mostly due to the inadequacy of existing disease and screening models. Furthermore, off-target drug effects and toxicity are not routinely screened for as they may impose more costs on the drug development process. A wide range of commonly prescribed drugs, including chemotherapeutic agents, antimicrobials, cardiovascular medications, psychotropics, and anticonvulsants, can induce peripheral nervous system (PNS) disorders by directly causing neuropathy, disrupting neurotransmission, or interacting with receptors expressed in target tissues. In vivo, the autonomic nervous system (ANS) regulates the function of nearly all organs in the body. However, innervation is neglected in developing most biofabricated tissue models. The ANS dysfunction can lead to many disorders.

There are only a few studies on tissue innervation with sensory neurons (and not ANS) where neurons and epithelial tissue are cultured in separated compartments connected to each other through intricate microchannels and grooves in microphysiological system (MPS) devices (FIG. 1). This form of innervation is mostly two-dimensional (2D) as nerve cells extend neurites in a planar monolayer with some cell body transmigration to the epithelial channel which is not physiologic. The present application fills a long-standing need for three-dimensional innervation for three-dimensional innervation of biofabricated tissues for disease modeling and drug development.

### SUMMARY

One aspect of the present application relates to a method for three-dimensional innervation of biofabricated tissue, comprising: culturing neurons or neuron progenitor cells on a surface to form cultured neurons, wherein the surface is treated with an extracellular matrix (ECM) protein and/or oxygen plasma treatment; casting a hydrogel layer on top of the cultured neurons, wherein the hydrogel layer has a thickness equal to or less than the maximum ability of neurons to vertically extend their neurites in a specific culture time period; and culturing a target tissue on top of the hydrogel layer to allow neurites from the neurons to extend through the hydrogel and innervate the target tissue.

Another aspect of the present application relates to a method for three-dimensional innervation of biofabricated tissue, comprising: treating a surface of a microporous nanomembrane with an extracellular matrix (ECM) protein and/or oxygen plasma to form a treated surface; culturing neurons on the treated surface of the microporous nanomembrane; casting a hydrogel layer on a surface opposite to the treated surface of the microporous nanomembrane; placing a double-sided open microbubble array on top of the hydrogel layer; and culturing a target tissue in the double-sided open microbubble array, wherein neurites from the neurons extend through the microporous nanomembrane and the hydrogel layer to innervate the target tissue.

Another aspect of the present application relates to three-dimensionally innervated biofabricated tissues produced by the method of the present application.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings. The figures herein are for illustrative purposes only and are not necessarily drawn to scale.

FIG. 1 shows two-dimensional innervation of epithelial tissue with sensory neurons.

FIGS. 2A-2B shows three-dimensional innervation in a standard multi-well plate (FIG. 2A) and a MPS device equipped with a microporous nanomembrane (FIG. 2B).

FIGS. 3A-3C shows monolayer culture of differentiated PC-12 cells on the underside of a membrane in the MPS. Day 4 culture at 4× (FIG. 3A), day 4 culture at 20× (FIG. 3B) with obvious neurites extension, day 6 culture at 20× (FIG. 3C) stained with Calcein AM for whole cell staining (green) and DAPI for cell nucleus (blue).

FIG. 4 shows 40× image of PC-12 3D neurite extension in Collagen (2 mg/ml)-MATRIGEL® (Corning Incorporated, Corning, NY) (4 mg/ml) complex hydrogel on day 5.

FIGS. 5A-5B shows a schematic (not to scale) of a double-sided open microbubble array (FIG. 5A). Spheroid/organoid tissue innervation in a MPS device (FIG. 5B).

FIGS. 6A-6F show hydrogel engineering and 3D tissue innervation in multi-well plates. (FIG. 6A) 3D neurite extension in hydrogel casts in a standard 96-well plate. (FIG. 6B) Maximum vertical neurite extension of different gel casts on day 7; (p<0.05). (FIG. 6C) Day 7 SG spheroid culture on Collagen I-MATRIGEL (2 mg/ml-4 mg/ml, so-called C2M4) hydrogel cast (20,000 cells/well) in a 96-well plate. (FIG. 6D) 3D view of the vertical 3D innervation of nerves in the hydrogel in PC12—salivary gland co-culture in a customized 96-well plate (FIGS. 6E and 6F). Innervation of salivary gland spheroids by sympathetic neurons. EpCAM labels salivary gland tissue mimetics (SGm) tissue, TUBB3 marks neurites, and Hoechst stains nuclei.

FIGS. 7A-7E show innervation of salivary gland cells separated from PC12 cells by a membrane. (FIG. 7A) 3 micron pores membrane with 20% porosity. (FIGS. 7B and 7C) Innervation of salivary gland cells by sympathetic neurons using the MPS with a 3 micron pore membrane, where PC12 cells are cultured on the underside of the membrane and salivary gland cells are cultured on the other side of the membrane. TUBB3 marks neurites, and Hoechst stains nuclei of both PC12 and salivary gland cells. (FIGS. 7D and 7E) Innervation of salivary gland cells by sympathetic neurons using the MPS with a 3 micron pore membrane, where PC12 cells are cultured on the underside of the membrane and salivary gland cells are cultured on the other side of the membrane. TUBB3 marks neurites, EpCAM labels SGm tissue and Hoechst stains nuclei of both PC12 and salivary gland cells.

FIGS. 8A-8C show 3D innervation of salivary gland cells separated from PC12 cells by a membrane and a hydrogel layer on top of the membrane. (FIG. 8A) 3D view of the vertical 3D innervation of SGm in the MPS with a 3 micron pore membrane, where PC12 cells are cultured on the underside of the membrane and salivary gland cells are cultured on top of a hydrogel layer on the other side of the membrane (FIGS. 8B and 8C). 3D Innervation of SGm by sympathetic neurons. TUBB3 marks neurites, EpCAM labels SGm tissue, and Hoechst stains nuclei of both PC12 and salivary gland cells. 3D neurite projection is depicted by dashed lines.

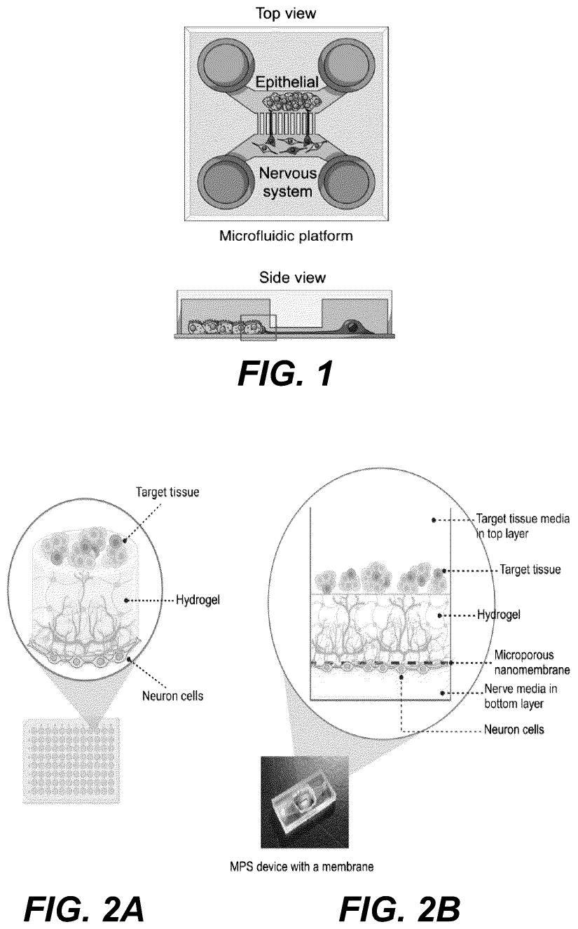

### DETAILED DESCRIPTION

The aspects of the application are described in conjunction with the exemplary embodiments, including methods, materials and examples, such description is non-limiting, and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

All publications and patents cited in this specification are cited to disclose and describe the methods and/or materials in connection with which the publications are cited. All such publications and patents are herein incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### I. Definitions

As used herein, the following terms have the meanings ascribed to them unless specified otherwise.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The phrase, “central nervous system (CNS)” consists of the brain and spinal cord, acting as the body's primary control center for processing information, regulating bodily functions, and coordinating movement.

The term “double-sided open microbubble array,” as used herein, refers to a, structure containing microbubble-cavities or wells open on both sides. In some embodiments, double-sided open microbubble array contains micron-scale spherical cavities (or wells) designed for cell culture. Double-sided open microbubble array may be fabricated from polymeric materials, such as polydimethylsiloxane (PDMS).

The term “extracellular matrix (ECM),” as used herein, refers to is a complex network of proteins (like collagen, fibronectin, laminin) and carbohydrates secreted by cells, forming a structural scaffold that supports, connects, and organizes tissues, while also regulating crucial cell functions like growth, movement, communication, and differentiation, acting as a dynamic microenvironment vital for tissue health and repair. ECM provides physical support, transmits signals, and helps cells adhere to each other, with its composition varying greatly by tissue, from the soft ECM in lungs to the calcified ECM in bone.

The term “ECM proteins” as used herein, refer to a diverse, complex network of secreted molecules that form a structural scaffold for cells in tissues. ECM proteins are essential for tissue integrity, cell adhesion, migration, proliferation, and signaling. Examples of ECM proteins include, but are not limited to, collagens (e.g., type I, II, III, IV and V, which are the most abundant structural proteins in ECM, providing tensile strength to skin, tendons, bone, and cartilage; Type IV collagen is also a key component of the basement membrane), elastin (a highly elastic protein, often associated with collagen, that allows tissues like blood vessels and lung tissue to stretch and recoil), fibronectin (a glycoprotein that aids in cell adhesion, migration, and wound healing), laminins (major proteins in the basement membrane that support cell differentiation and migration), tenascins (glycoproteins (TN-C, TN-R, TN-W, TN-X, TN-Y) involved in tissue remodeling and cell adhesion, particularly during development and injury repair), proteoglycans (e.g., aggrecans, which are molecules that create a hydrated gel, providing cushioning and resistance to compression), and fibrillins (proteins essential for the formation of elastic fibers.

The term “hydrogel,” as used herein, refers to a three-dimensional, cross-linked network of hydrophilic polymer chains that can absorb and retain large amounts of water (up to 99% of their weight). Hydrogels act as soft, porous, and flexible materials that mimic natural tissues, and are widely used in wound dressings, drug delivery systems, personal hygiene products, and contact lenses. Examples of hydrogels include, but are not limited to, hydrogels formed from naturally existing molecules (natural hydrogels), hydrogels formed from synthetic molecules (synthetic hydrogels), and hydrogels formed from a mixture of naturally existing molecules and synthetic molecules (hybrid hydrogels).

Examples of natural hydrogels include, but are not limited to, hydrogels formed from collagen, gelatin, fibrin, chitosan, alginate and cellulose. Examples of synthetic hydrogels include, but are not limited to, hydrogels formed from polyethylene glycol (PEG), poly(vinyl alcohol) (PVA), polyacrylamide (PAAm) and poly(N-isopropylacrylamide) (PNIPAAm). Examples of hybrid hydrogels include, but are not limited to, hydrogels formed from or comprising one or more of hyaluronic acid-PEG (HA-PEG), arginine-glycine-aspartate (RGD)-alginate-conjugated PEG (RGD-alginate-PEG, and (Collagen-PEG-Laminin). Examples of commercially available hydrogels include, but are not limited to, MATRIGEL (comprising primarily laminin and collagen IV) and collagen-MATRIGEL complex (a composite, three-dimensional hydrogel combining collagen type I and MATRIGEL).

The term “micropore membrane,” as used herein, refers to a thin, solid material containing an interconnected network of tiny pores, slits or mixtures thereof that have a size that is large enough to allow neurite extensions (e.g., axons and dendrites) to pass through the membrane, but not large enough to allow the cell body of a neuron (soma) to pass through.

The term “microphysiological systems (MPS),” “tissue chip,” and “organ-on-a-chip refer to advanced microfluidic devices that culture human or animal cells in 3D environments, simulating tissue architecture, mechanical cues, and fluid flow to better mimic organ function compared to 2D cell cultures. These microfluidic devices may contain human/animal cells grown on a scaffold to mimic the structure, function, and physiological response of human/animal organs like the heart, liver, or lung, and may be used to test drug safety, model diseases, and study human physiology more accurately than animal models.

The term “neurotropic factors,” as used herein, refers to proteins essential for the survival, development, and function of neurons. Neurotropic factors are critical for repairing damage, maintaining nervous system health, and organ tissue health and function. Examples of neurotropic factors include, but are not limited to, nerve growth factors (NGF), beta-NGF (NGFβ); brain-derived neurotrophic factor (BDNF), neurotrophin-3 (NT-3) and neurotrophin-4/5 (NT-4/5) and glial cell line-derived neurotrophic factor (GDNF). The term also include other types of growth factors, such as fibroblast growth factors (FGF), insulin-like growth factor I (IGF-I) and transforming growth factor-beta (TGF-β) that may have an effect on neuron growth.

The terms “neuron” and “nerve cell” are used interchangeably and refer to an excitable cell that fires electric signals called action potentials across a neural network in the nervous system. Neuron stimulation induces calcium flux in the cells. A neuron is the fundamental structural and functional unit of the nervous system. It is designed to receive, process, and transmit information through electrical and chemical signals. Neurons include four structural types: multipolar (most common, one axon, many dendrites), bipolar (one axon, one dendrite), unipolar/pseudo-unipolar (single process splits into axon and dendrite), and sometimes anaxonic (no true axon). Functionally, neurons are categorized as sensory (input), motor (output), and interneurons (connecting), while supporting cells called glial cells (astrocytes, oligodendrocytes, microglia, Schwann cells) also form crucial parts of nerve tissue. A typical neuron (or nerve cell) consists of three main parts: (1) soma (Cell Body), which is the central part of the neuron that contains the nucleus and organelles and maintains the cell's structure and provides energy; (2) dendrites, which are branch-like extensions that receive incoming messages from other neurons or the environment and carry them toward the cell body; and (3) axon, which is a long, tail-like projection that carries electrical impulses (action potentials) away from the cell body toward other neurons, muscles, or glands.

The term “neurites,” as used herein, refers to any projections (cytoplasmic extensions) from the cell body of a neuron, serving as the precursors to axons and dendrites.

The term “optional” or “optionally” means that the subsequent described event, circumstance or substituent may or may not occur, and that the description includes instances where the event or circumstance occurs and instances where it does not.

The term “organoids,” as used herein, refers complex clusters of organ-specific cells, such as those from the stomach, liver, or bladder. Organoids are made of stem cells or progenitor cells and self-assemble when given a scaffolding extracellular environment, such as a hydrogel matrix.

The term “peripheral nervous system” (PNS), as used herein, refers to all nerves outside the brain and spinal cord, functioning as a communication network connecting the CNS to limbs, organs, and sensory receptors. PNS enables voluntary movement (somatic) and involuntary, automatic functions (autonomic), such as heart rate and digestion. PNS may subdivided into the somatic nervous system (SNS), which controls voluntary movements of skeletal muscles and transmits sensory information to the CNS, and autonomic nervous system (ANS), which manages involuntary functions (heart, smooth muscle, glands). ANS neurons typically use a two-neuron chain: the preganglionic neuron (myelinated) extends from the CNS to a peripheral ganglion, where it synapses with the postganglionic neuron (unmyelinated), which then innervates the effector organ.

The term “primary cells,” as used herein, refers to specialized, mature cells taken directly from living tissue with a finite lifespan and limited expansion.

The term “primary neuron cells,” as used herein, refers to nerve cells isolated directly from living tissue (animal or human).

The term “sensory neurons (or afferent neurons)” are specialized nerve cells that convert external or internal stimuli—such as light, sound, touch, or chemical signals—into electrical impulses transmitted to the CNS. Examples of sensory neurons include, but are not limited to, photoreceptors in the eye, olfactory neurons for smell, mechanoreceptors for touch, and nociceptors for pain.

The term “spheroids,” as used herein, refers to simple clusters of cells, such as from tumor tissue, embryoid bodies, hepatocytes, nervous tissue, or mammary glands. The cells form spheroids by simply sticking to each other.

The term “stem cells,” as used herein, refers to undifferentiated, renewable cells capable of self-renewal and differentiation into various specialized cell types. Examples of stem cells include, but is not limited to, pluripotent stem cells such as embryonic stem cells (ESC), and induced pluripotent stem cells (iPSC), and multipotent (adult) stem cells such as hematopoietic stem cells (blood cells), mesenchymal stem cells (bone, fat, cartilage), neural stem cells (neurons, glia), and dental pulp stem cells.

The term “tissue mimetics” as used herein refers to an aggregation of cells that are morphologically or functionally related, or cell systems derived from ex vivo human/animal tissues/organs. Thus, in vitro cultured cells, as well as tissues, organs, and the like, are encompassed by the term tissue or tissue mimetic.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’.”

It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

The recitation of numerical ranges by endpoints includes all numbers and fractions subsumed within the respective ranges, as well as the recited endpoints.

### II. Method for Three-Dimensional Innervation of Bio-Fabricated Tissues

The present application addresses a key translational barrier in disease modeling and drug development by teaching methods to innervate biofabricated tissues. The present application describes methods to innervate bio-fabricated tissues in three dimensions (3D). Innervation in many organs, including salivary glands, eyes, heart, skeletal muscles, lungs, kidneys, and gastrointestinal (GI) tract, that are affected by the PNS can be modeled using the disclosed platform. Accordingly, these models can be used for disease study and high-throughput drug/toxicity screening.

One aspect of the present application relates a method for three-dimensional innervation of biofabricated tissue. The method comprises the steps of culturing seeding neurons on a culturing surface, wherein the culturing surface is treated with a cell attachment enhancing agent or oxygen plasma treatment, casting a hydrogel layer on top of the cultured neurons, and culturing a target tissue on top of the hydrogel layer to allow neurites from the cultured neurons to extend through the hydrogel and innervate the target tissue.

In some embodiments, the culturing surface is the bottom surface of a tissue culturing well or tissue culturing plate. In some embodiments, the method further comprises the step of placing a micropore or nanoporous membrane on top of cultured neurons prior to the casting of the hydrogel layer.

In some embodiments, the culturing surface is one side (e.g., the underside) of a micropore membrane of a MPS device and the hydrogel layer is casted on an opposite side (e.g., the upperside) of the micropore membrane.

Another aspect of the present application relates to a method for three-dimensional innervation of biofabricated tissue. The method comprises the steps of treating a surface of a micropore membrane with a cell attachment enhancing agent or oxygen plasma treatment to form a treated surface; culturing neurons on the treated surface of the micropore membrane to allow the seeding neurons to attach to the micropore membrane; casting a hydrogel layer on a surface opposite to the treated surface of the micropore membrane; placing a double-sided open microbubble array on top of the hydrogel layer; and culturing a target tissue in the double-sided open microbubble array, wherein neurites from the seeding neurons extend through the micropore membrane and the hydrogel layer to innervate the target tissue in the double-sided open microbubble array.

### Cell Attachment Enhancing Agents and Oxygen Plasma Treatment

The cell attachment enhancing agent can be any agent that enhances attachment of the seeding neurons to the surface. In some embodiments, the cell attachment enhancing agent comprises an ECM protein. Examples of ECM proteins include, but are not limited to, collagens (e.g., type I, II, III, IV and V), elastin, fibronectin, laminins, tenascins (glycoproteins (TN-C, TN-R, TN-W, TN-X, TN-Y), proteoglycans and fibrillins. In some embodiments, the cell attachment enhancing agent comprises a synthetic polymer, such as poly-L-Lysine and polydopamine (PDA). In some embodiments, the cell attachment enhancing agent is an ECM protein selected from the group consisting of collagen I, fibronectin and collagen IV.

The surface treatment for both cell seeding and hydrogel sealing can be performed by plasma treatment of oxygen.

### Seeding Cells

The seeding cells can be neurons or any cell type capable of differentiating into neurons. In some embodiments, the seeding cells are primary nerve cells. In some embodiments, the seeding cells are neural tissue-derived cell lines. In some embodiments, the neural tissue-derived cell lines are animal neural tissue-derived cell lines such as, but not limited to, PC12 cells. In some embodiments, the neural tissue-derived cell lines are human neural tissue-derived cell lines. Examples of human neural tissue-derived cell lines include, but are not limited to, SH-SY5Y, LUHMES, ReNcell VM (Ventral Mesencephalon) and CX (Cortex), NT2 (NTERA-2 cl.D1), SK-N-SH/CHP-134, SK-N-SH, IMR-32, BE(2)-M17 cells. In some embodiments, the seeding cells are human pluripotent stem cell (hPSC)-derived neural crest cells (NCCs), where they can differentiate to the respective PNS system and glial cells by the neurotropic factors received from a co-culture with an effector tissue. In some embodiments, the seeding cells are nerve cells derived from, or are induced to differentiate into, PNS nerve cells. In some embodiments, the seeding cells are nerve cells derived from, or are induced to differentiate into, CNS nerve cells. In some embodiments, the seeding cells are nerve cells derived from, or are induced to differentiate into, ANS nerve cells. In some embodiments, the seeding cells are nerve cells derived from, or are induced to differentiate into, SNS nerve cells.

### Culturing Conditions for Seeding Neurons

The seeding neurons are cultured in a nerve medium under standard cell culture conditions and for a period of time sufficient to allow attachment of the seeding neurons to the culturing surface. In some embodiments, the seeding neurons are cells from a neural tissue-derived cell line and are treated with nerve growth factor to grow and differentiate. In some embodiments, the seeding neurons are cultured in the absence of any exogenously added neurotropic factors. In some embodiments, the seeding neurons are cultured in the presence of exogenously added neurotropic factors.

Examples of the nerve media include, but are not limited to, base culture and maintenance media like DMEM (Dulbecco's Modified Eagle Medium) and RPMI 1640 with the absence or very low amount of serum.

Examples of cell culture conditions include, but are not limited to, base culture and maintenance media like DMEM (Dulbecco's Modified Eagle Medium) and RPMI 1640 with an appropriate amount of serum. Cell cultures are maintained in standard incubators at 37° C. with 5% CO₂. Tri-gas incubators can be used to vary, for example the 02 levels if desired.

In some embodiments, the seeding neurons are cultured for a period of 12 hours to 7 days before the casting of hydrogels and introducing the target (effector) tissue for co-culture.

### The Hydrogel Layer

The hydrogel layer may be a layer of natural hydrogel, synthetic hydrogel or hybrid hydrogel. In some embodiments, the hydrogel layer comprises a layer of natural hydrogel. In some embodiments, the layer of natural hydrogel comprises laminin, fibrin, collagen I or MATRIGEL. In some embodiments, the hydrogel layer comprises a layer of synthetic hydrogel. In some embodiments, the synthetic hydrogel comprises polyethylene glycol (PEG), poly(vinyl alcohol) (PVA), polyacrylamide (PAAm) or poly(N-isopropylacrylamide) (PNIPAAm). In some embodiments, the hydrogel layer comprises a layer of hybrid hydrogel. In some embodiments, the hybrid hydrogel comprises one or more of hyaluronic acid-PEG (HA-PEG), arginine-glycine-aspartate (RGD)-alginate-conjugated PEG (RGD-alginate-PEG), and Collagen-PEG-Laminin.

In some embodiments, the hydrogel layer has a thickness equal to, or less than, the maximum ability of neurons to vertically extend their neurites. In some embodiments, the hydrogel layer has a thickness in the range of 0-1000, 50-5000, 50-2000, 50-1000, 50-500, 50-200, 100-5000, 100-2000, 100-1000, 100-500, 100-200, 200-5000, 200-2000, 200-1000, 200-500, 500-5000, 500-2000, 500-1000, 1000-5000, 1000-2000 or 2000-5000 micron.

### The Target Tissue

The target tissue can be any tissue that can be innervated with the seeding neurons. In some embodiments, the target tissue is derived from a tissue innervated with the PNS. In some embodiments, the target tissue is formed by culturing primary cells, stem cells or cell lines on the hydrogel layer. In some embodiments, the target tissue is derived from a tissue/organ selected from the group consisting of salivary glands, heart, eye, skeletal muscles, lungs, kidney and gastrointestinal (GI) tract. In some embodiments, the target tissue is cultured in the absence of exogenously added neurotropic factors like NGF.

### The Micropore Membrane

The micropore membrane can be any porous membrane with pore sizes that allow passage of neurites from the cultured seeding neurons but would not allow passage of the cell body of the cultured seeding neurons. In some embodiments, the micropore membrane comprises a polymer selected from the group consisting of polytetrafluoroethylene (PTEE), polyurethane (PU), polypropylene, and polyamides. In some embodiments, the micropore membrane has a pore size in the range of 0.1-10, 0.1-5, 0.1-2, 0.1-1, 0.1-0.5, 0.1-0.2, 0.2-10, 0.2-5, 0.2-2, 0.2-1, 0.2-0.5, 0.5-10, 0.5-5, 0.5-2, 0.5-1, 1-10, 1-5, 1-2, 2-10, 2-5, 5-10, 1-20 microns based on IUPAC standards for porous materials. In some embodiments, the micropore membrane has a pore size in the range of 0.5-2 microns based on IUPAC standards for porous materials. In some embodiments, the micropore membrane has a pore size of about 1 micron based on IUPAC standards for porous materials. In some embodiments, the micropore membrane has a thickness in the range of 0.1-100 microns.

### The Double-Sided Open Microbubble Array

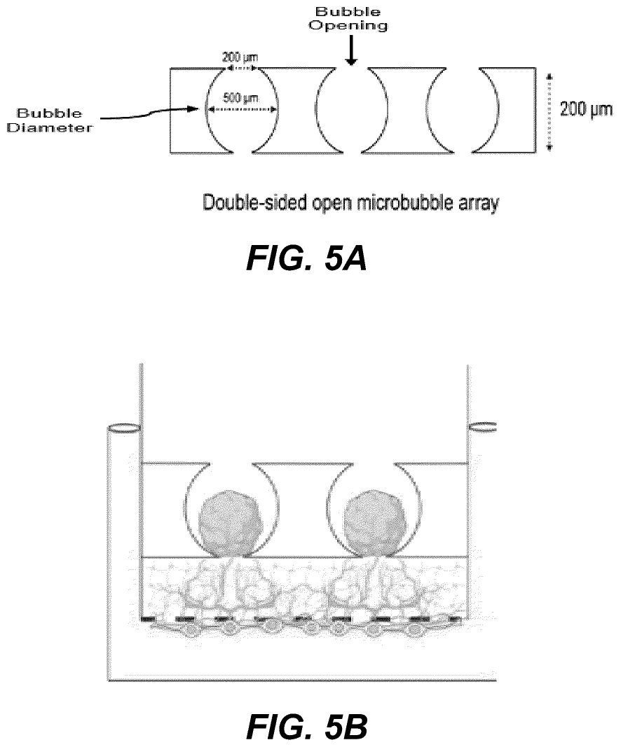

The double-sided open microbubble array can be a double-sided open microbubble membrane. In some embodiments, the double-sided open microbubble array has a bubble diameter (as shown in FIG. 5A) in the range of 100-2000 microns and a bubble opening (as shown in FIG. 5A) size in the range of 100-1000 microns. In some embodiments, the double-sided open microbubble array or membrane has a thickness of 20-2000 microns.

In some embodiments, the double-sided open microbubble array is bonded to the nanomembrane by a pressure sensitive adhesive (PSA). The hydrogel can be injected to the gap created between the membrane and microbubble array through one of the double-sided open MBs over the frame of the membrane.

In some embodiments, the target tissue is formed in the double-sided open microbubble array by seeding target cells in the double-sided open microbubble array and culturing seeded target cells in the double-sided open microbubble array to form innervated target tissue. In some embodiments, the target tissue is formed in the double-sided open microbubble array by seeding target cells in the double-sided open microbubble array, and culturing the seeded target cells in the double-sided open microbubble array to form innervated spheroids or organoids.

### Culturing Conditions for Target Tissue

The target tissue is cultured in a target tissue medium under standard tissue culture conditions and for a period of time sufficient to allow attachment of the seeding neurons to the culturing surface. In some embodiments, the target tissue forms spheroids or organoids. In some embodiments, the target tissue is cultured in the absence of any exogenously added neurotropic factors.

Examples of the target tissue media include, but are not limited to, salivary glands, heart, lung, skin, liver, pancreas, kidney, brain, base culture and maintenance media like DMEM (Dulbecco's Modified Eagle Medium), F12, and RPMI 1640 with an appropriate amount of serum and tissue-specific culture elements.

Examples of target tissue culture conditions include, but are not limited to, base culture and maintenance media like DMEM (Dulbecco's Modified Eagle Medium), F12, and RPMI 1640 with an appropriate amount of serum and tissue-specific culture elements. Cell cultures are maintained in standard incubators at 37° C. with 5% CO₂. Tri-gas incubators can be used to vary, for example the 02 levels if desired.

In some embodiments, the target tissue is cultured for a period of 12 hours to 21 days.

### III. Bio-Fabricated Tissues

Another aspect of the present application relates to 3D-innervated bio-fabricated tissues produced with the method of the present application. These tissues can be used for disease study and high-throughput drug/toxicity screening.

The present application is further illustrated by the following examples that should not be construed as limiting. The contents of all references, patents, and published patent applications cited throughout this application, as well as the Figures and Tables, are incorporated herein by reference.

### EXAMPLES

### Example 1: 3D Innervation Experimental Design

An example of 3D innervation of a tissue in a multi-well plate and a MPS equipped with a nanomembrane are illustrated in FIGS. 2A and 2B, respectively. In multi-well plates, the neurons can be cultured on the surface of a well-treated with an extracellular matrix (ECM) protein including, collagen I (125 μg/ml), collagen IV, and fibronectin to enable the cells to attach to the surface (FIG. 2A). A hydrogel (e.g. natural hydrogels like laminin, collagen I, MATRIGEL, collagen I-MATRIGEL complex, and synthetic hydrogels like polyethylene glycol (PEG) and their mixture complex) is cast on top of the neurons with a thickness equal to or less than the maximum ability of neurons to vertically extend their neurites in 3D. The target tissue is cultured on top of the gel. The hydrogel and nervous system are engineered to project upward through the hydrogel to innervate and affect the target tissue function. Human pluripotent stem cell (hPSC)-derived neural crest cells (NCCs), Primary neuron cells from animals and humans, differentiated stem cells to neurons including sensory, parasympathetic, and sympathetic neurons can be used to innervate the target tissues. PC-12 (ATCC CRL-1721) cell line was used as a neuron model. The cells can be differentiated into sympathetic neurons upon treatment with nerve growth factor (NGF, 100 ng/ml). Mouse extracted salivary gland cells were cultured on top of the gel with no NGF in the culture media and differentiated the PC12 cells to neurons and boosted the sympathetic neurite projection into the gel by continuously producing fresh NGF. Z stack images of the gel thickness from the bottom (nerve cell bodies)-up toward (neurite extension and salivary gland spheroids on top of the gel) showed the potential of 3D tissue innervation.

### Example 2: Membrane Separation

In an embodiment, the nerve cells are cultured on the underside of a membrane in transwell plate format to ensure that the cell body (soma) cannot transmigrate through the microporous membrane to enter the gel. A hydrogel and the target tissue are cultured on the topside of the membrane. Membrane with pores (0.4-8 microns) can be used to favor neurite/axon extension while inhibiting the cell body from transmigration.

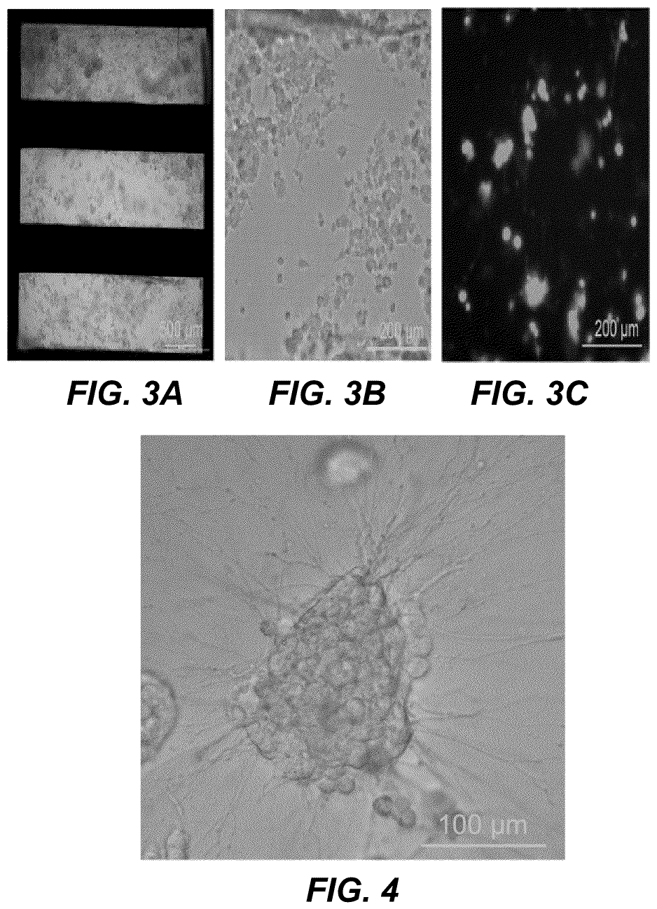

In a more advanced embodiment, the PC12 cells are cultured on the underside of a nanomembrane (FIG. 2B) that is engineered to ensure that the cell body (soma) cannot transmigrate through the nanomembrane to enter the gel. In a preliminary test, PC12 cells cultured on the underside of a membrane maintained their viability and showed neurite extension (FIG. 3). The nanomembrane can be engineered with micro pores or microslits where the nerve cells are cultured on the underside of the membrane. A hydrogel and the target tissue are cultured on the topside of the nanomembrane. The pores (preferred size of ~1 micron) of the membrane are engineered to favor neurite/axon extension while inhibiting the cell body from transmigration. SiMPore manufactured membranes can be used for this purpose in the modular μSiM (microphysiological system featuring a Silicon Membrane) cartridge.

Preferred embodiments optimize the hydrogel in terms of pore size, porosity, chemical composition, swelling ratio and mechanical strength, stiffness, and elasticity that favors both vertical 3D neurite extension and target tissue formation and function in a biorelevant condition. Different gels including collagen I only, MATRIGEL only, Collagen I-MATRIGEL complex hydrogels at different concentrations/compositions, PEG, and PEG-Collagen/MATRIGEL complex hydrogels at different concentrations/compositions are being tested and engineered for the specified application. In a preliminary tests a preferred composition of, Collagen (2 mg/ml)-MATRIGEL (4 mg/ml) complex hydrogel promoted neurite 3D extension (FIG. 4) with a maximum vertical projection equal to 300 μm in a 96-well plate platform on day 7.

### Example 3: MPS Format

FIGS. 5A-5B show a schematic (not to scale) of a double-sided open microbubble array (FIG. 5A) and spheroid/organoid tissue innervation in a MPS device (FIG. 5B).

### Example 4: Multi-Well Plate Format

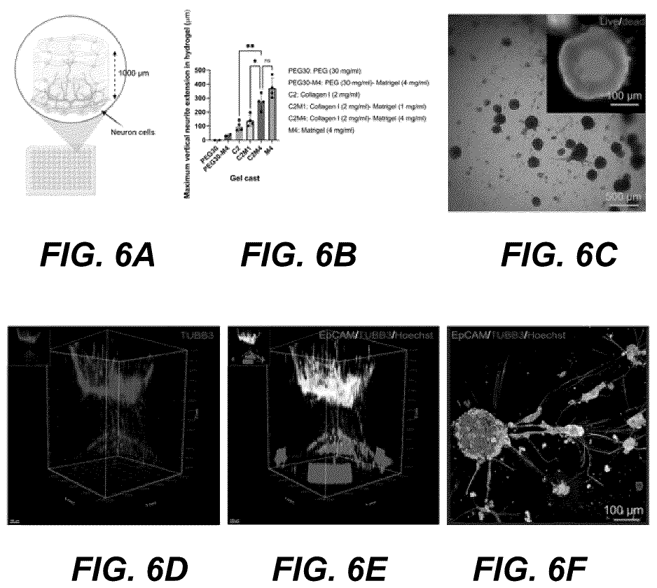

This study showed that chemical properties (biocompatibility, biodegradability, and biochemical cues), physical properties (density, porosity, anisotropy), and mechanical properties (Young's modulus, viscoelasticity) are critical components for in vitro tissue innervation. For convenience and high-throughput testing of different conditions, the hydrogel engineering was performed in commercial multi-well plates (FIG. 6A). PC12 (ATCC CRL-1721) cells were cultured on the surface of wells of a 96-well plate (5000 cells/well), pretreated with collagen I (125 μg/ml) to enable cell attachment. Different gels were cast on top of the cells with an approximate thickness of 1 mm, including MMP-degradable PEG, collagen I, MATRIGEL, and collagen I-MATRIGEL mixtures. Typically, soft hydrogels with a Young's modulus of <1 KPa are reported to be optimal for neural maturation and extension. The cells were differentiated into sympathetic neurons with the nerve growth factor (NGF) at 100 ng/ml in the media for 7 days. Z-stack images of the gel cast taken on day 7 from bottom (nerve cell bodies)-up toward the top of the gel showed that MATRIGEL (4 mg/ml) and Collagen I-MATRIGEL (2 mg/ml-4 mg/ml, so-called C2M4) with pores up to 10 μm have the maximum vertical neurite extension in the gel (FIG. 6B).

In addition, the hydrogel must have sufficient stiffness to provide a base for target (effector) tissue culture and exhibit a low swelling ratio in case of being used with double sided open microbubble (MB) array to prevent projection into the MB. Preliminary data showed that MATRIGEL is too soft and swells a lot; in a way, it is impossible to measure its swelling ratio. However, C2M4 showed a swelling ratio of 1.05±0.25. In a preliminary test, C2M4 hydrogel cast in a 96-well plate supported salivary gland tissue mimetics (SGm) formation (FIG. 6C). In a co-culture of PC-12 and SGm with the optimized cast hydrogel in between, and with no NGF in media, the vertical 3D innervation of nerves in the hydrogel (FIGS. 6D and 6E) and connection, i.e. innervation of SGm, was observed (FIGS. 6E and 6F). Salivary gland spheroids produce a range of neurotropic factors, including NGF. Continuous SGm produced NGF, differentiated PC-12 cells into a wide network of sympathetic neurons (a division of the ANS), and intensified the vertical neurite extension for up to 600 μm, demonstrating the value of multi-tissue neuroeffector signaling for human-relevant tissue innervation. However, nerve cell bodies' migration through gel and not spatially limited SGm spheroid formation with necrotic cores, remained the challenges that can be addressed in the hybrid MPS with the microporous membrane with or without the Microbubble array.

Different cell types, including ARPE-19 as human retinal pigment epithelial (RPE) cell line, Skin primary keratinocytes, and Lung slices, were used as target (effector) tissues on top of the hydrogel, and all differentiated PC12 cells to neurons and initiated 3D innervation through the hydrogel. They are all reported to produce NGF (reference: The Human Protein Atlas).

### Example 4: MPS Device with a Microporous Membrane

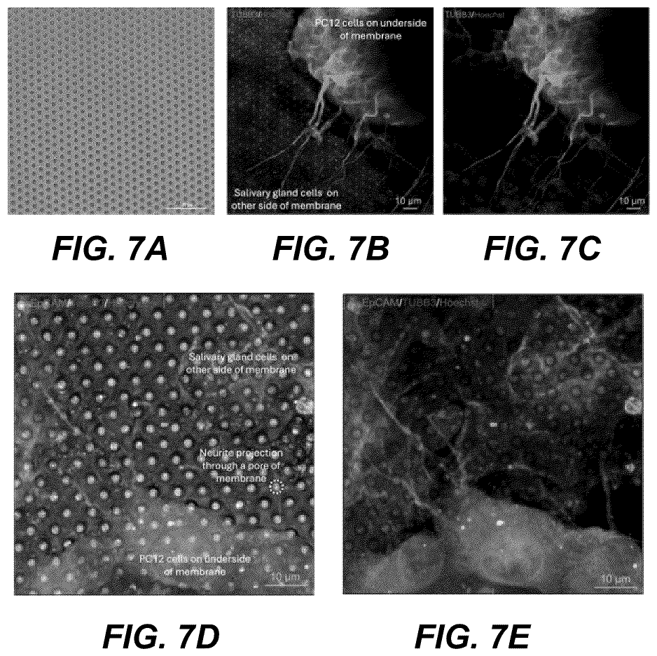

Current platforms often do not replicate the in vivo configuration of the peripheral nervous system, where nerve cell bodies are contained in distinct ganglia and project axons over distances toward target tissues. Herein, a membrane-equipped MPS platform is used to keep the nerve bodies in the nerve compartment separated from the target tissue compartment by a membrane. The platform will allow 3D projection of neurite/axon (with a diameter between 0.15 to 2.0 μm) through the pores of the membrane. Commercial SiMPore, Inc. membranes were used for this purpose. The membrane with 3 micron pores (20% porosity) (FIG. 7A) was found to completely inhibit cell body transmigration while allowing neurite extension toward the target tissue. The optical transparency of the nanomembrane allows in situ analysis of two tissue compartments and neurite/axon extensions without disassembly of the device. The MPS platform with a 3 micron pore membrane was used to model salivary gland tissue innervation (FIGS. 7B-E). Differentiated PC12 cells to sympathetic neurons projected their neurites through the membrane pores and connected to the salivary gland target tissue.

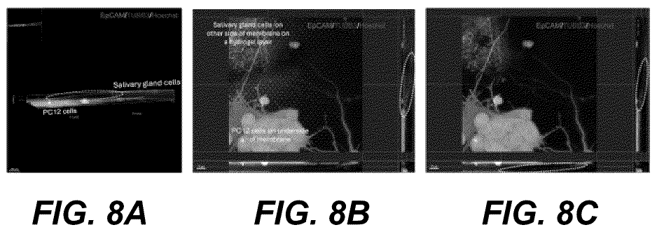

Adding a hydrogel layer between the nerve cells and the target tissue, the MPS with 3 micron pores showed 3D tissue innervation over a distance of ~100 μm in a co-culture of PC12 and primary salivary gland cells without addition of any neurotropic factors including NGF in the media (FIGS. 8A-8C). Differentiated PC12 to sympathetic neurons projected their neurites through the membrane pores and hydrogel and connected to the salivary gland target tissue on top of the hydrogel.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

What is claimed is:

1. A method for three-dimensional innervation of biofabricated tissue, comprising:
culturing neurons or neuron progenitor cells on a surface to form cultured neurons, wherein the surface is treated with an extracellular matrix (ECM) protein and/or oxygen plasma treatment;
casting a hydrogel layer on top of the cultured neurons, wherein the hydrogel layer has a thickness equal to or less than the maximum ability of neurons to vertically extend their neurites in a specific culture time period; and
culturing a target tissue on top of the hydrogel layer to allow neurites from the neurons to extend through the hydrogel and innervate the target tissue.

2. The method of claim 1, wherein the ECM protein is collagen I, fibronectin, or collagen IV.

3. The method of claim 1, wherein the hydrogel is a natural hydrogel comprising laminin, fibrin, collagen I, collagen IV, MATRIGEL, or mixtures thereof.

4. The method of claim 1, wherein the hydrogel is a synthetic hydrogel comprising polyethylene glycol (PEG), poly(vinyl alcohol) (PVA), polyacrylamide (PAAm) or poly(N-isopropylacrylamide) (PNIPAAm).

5. The method of claim 1, wherein the hydrogel is a hybrid hydrogel comprising one or more of hyaluronic acid-PEG (HA-PEG), arginine-glycine-aspartate (RGD)-alginate-conjugated PEG (RGD-alginate-PEG), and Collagen-PEG-Laminin.

6. The method of claim 1, wherein the target tissue is derived from a tissue innervated with the peripheral nervous system (PNS).

7. The method of claim 7, wherein the target tissue is derived from a tissue/organ selected from the group consisting of salivary glands, heart, eye, skeletal muscles, lungs, kidney and gastrointestinal (GI) tract.

8. The method of claim 1, wherein the target tissue is derived from cells selected from the group consisting of cultured primary cells, cell-lines and isolated stem cells.

9. The method of claim 1, wherein the target tissue is cultured in the presence or absence of exogenously added neurotropic factors.

10. The method of claim 1, further comprising the step of placing a membrane, optionally a microporous nanomembrane, on top of cultured neuron cells, prior to the step of casting the hydrogel layer.

11. A three-dimensionally innervated biofabricated tissue produced with the method of claim 1.

12. A method for three-dimensional innervation of biofabricated tissue, comprising:
treating a surface of a microporous nanomembrane with an extracellular matrix (ECM) protein and/or oxygen plasma to form a treated surface;
culturing neurons on the treated surface of the microporous nanomembrane;
casting a hydrogel layer on a surface opposite to the treated surface of the microporous nanomembrane;
placing a double-sided open microbubble array on top of the hydrogel layer; and
culturing a target tissue in the double-sided open microbubble array, wherein neurites from the neurons extend through the microporous nanomembrane and the hydrogel layer to innervate the target tissue.

13. The method of claim 12, wherein the microporous nanomembrane has a pore size in the range of 0.1-8 micron based on IUPAC standards for porous materials.

14. The method of claim 13, wherein the microporous nanomembrane has a pore size in the range of 0.5-2 micron based on IUPAC standards for porous materials.

15. The method of claim 12, wherein the hydrogel layer has a thickness equal to or less than the maximum ability of neurons to vertically extend their neurites in the specific culture period.

16. The method of claim 15, wherein the hydrogel layer has a thickness in the range of 0-2000 microns.

17. The method of claim 12, wherein the double-sided open microbubble array has a bubble diameter in the range of 100-2000 micron and a bubble opening size in the range of 100-1000 micron.

18. The method of claim 12, wherein the double-sided open microbubble array has a thickness of 20-2000 micron.

19. The method of claim 12, wherein the double-sided open microbubble array is bonded to the membrane by a pressure sensitive adhesive (PSA) or plasma etch and hydrogel is injected through one of the microbubbles.

20. A three-dimensionally innervated biofabricated tissue produced with the method of claim 12.
